SEQUENCE LISTING (9 sequences)

SEQ ID NO: 1 - DNA, 120 bp, Artificial Sequence
  Synthetic sequence
atgaaaaatctttattttcaaggtatgcctgttggtattgttattgctcctaaaaaatctccttttacagctaaaaaacctggtcctgttctgtctggtgttaaagctggtcctggttaa

SEQ ID NO: 2 - DNA, 39 bp, Artificial Sequence
  Synthetic primer
aaaaagcaggcttcatggaaaatctttattttcaaggta

SEQ ID NO: 3 - DNA, 38 bp, Artificial Sequence
  Synthetic primer
agaaagctgggtcttaaccaggaccagctttaacacca

SEQ ID NO: 4 - DNA, 50 bp, Artificial Sequence
  Synthetic primer
ggggacaagtttgtacaaaaaagcaggcttcatggaaaatctttattttc

SEQ ID NO: 5 - DNA, 50 bp, Artificial Sequence
  Synthetic primer
ggggaccactttgtacacgaaagctgggtcttaaccaggaccagctttaa

SEQ ID NO: 6 - DNA, 181 bp, Artificial Sequence
  PCR amplification product
ggggacaagtttgtacaaaaaagcaggcttcatggaaaatctttattttcaaggtatgcctgttggtattgttattgctcctaaaaaatctccttttacagctaaaaaacctggtcctgttctgtctggtgttaaagctggtcctggttaagacccagctttcttgtacaaagtggtcccc

SEQ ID NO: 7 - DNA, 31 bp, Artificial Sequence
  Synthetic primer
ggggacaactttgtatagaaaagttgtcnnn

SEQ ID NO: 8 - DNA, 30 bp, Artificial Sequence
  Synthetic primer
ggggactgcttttttgtacaaacttgcnnn

SEQ ID NO: 9 - protein, 31 aa, Argopecten purpuratus
MPVGIVIAPKKSPFTAKKPGPVLSGVKAGPG

(19) United States
(12) Patent Application Publication
(10) Pub. No.: US 2013/0340124 A1
(43) Pub. Date: Dec. 19, 2013

(54) CHIMERIC GENE FOR HETEROLOGOUS EXPRESSION THAT ENCODES PEPTIDES WITH ANTIMICROBIAL ACTIVITY

(71) Applicant: INSTITUTO DE INVESTIGACIONES AGROPECUARIAS, Santiago (CL)

(72) Inventors: Christian Fernando Montes Serey, Santiago (CL); Humberto Godofredo Prieto Encalada, Santiago (CL); Gloria Maria Arenas Diaz, Santiago (CL); Eduardo Andres Tapia Rodriguez, Santiago (CL)

(73) Assignee: INSTITUTO DE INVESTIGACIONES AGROPECUARIAS, Santiago (CL)

(21) Appl. No.: 13/784,243

(22) Filed: Mar. 4, 2013

(30) Foreign Application Priority Data
Mar. 5, 2012 (CL) 593-2012

(51) Int. Cl. C07K 14/435

(52) U.S. Cl. CPC C07K 14/43504
USPC 800/301; 536/23.5; 530/300; 435/419

(57) ABSTRACT

A chimeric nucleotide sequence is provided encoding peptides with antimicrobial activity to be expressed in plants, plant cells or transformed plant material that will produce the peptide sequences derived from SEQ ID No. 1 and SEQ ID No. 6. A method is also provided for conferring resistance or tolerance to plant pathogenic fungi or bacteria on a plant using suitable transfer vectors, which contain the coding sequence for the peptides with antimicrobial activity.

### FIELD OF THE INVENTION

This invention relates to a chimeric nucleotide sequence encoding peptides with antimicrobial activity.

### BACKGROUND OF THE INVENTION

Antimicrobial peptides (AMP) are found in nature and have been isolated from several organisms, including animals and plants. In recent years, these molecules have shown important anti-pathogenic activity against Gram positive and Gram negative bacteria and fungi. AMPs are usually composed of 12-50 cationic and amphipathic amino acids (Broekaert et al., 1997; Zasloff, 2002, Marshall and Arenas, 2003). The natural AMPs are divided into groups characterized by peptides formed by β sheets, α-helices, extended structures and helix loop or loop structures, and of them, the first two are the most abundant (Dathe et al., 1999; Gao et al., 2000; Lehrer and Ganz, 2002; Bulet et al., 2004). The interaction between AMPs and their target cells is markedly influenced by factors such as the type and scope of their structure, cationicity, hydrophobicity, amphipathicity and amino acid sequence (Yeaman and Yount, 2003; Jenssen et al., 2006; Soltani et al., 2007). More than 2300 peptides have been described, and information on their structures, properties and mechanisms of action can be found in databases such as APD, ANTIMIC and AMPer.

Recently, AMPs have received attention as new substitutes for conventional pesticides and antibiotics as pathogens do not develop resistance to them because of their mechanism of action (Yeaman and Yount, 2003). However, the use of peptides as alternative drugs has encountered some difficulties, particularly the low recovery obtained after extraction from the tissue of origin. Therefore, their acquisition by chemical synthesis or the expression of the protein using a recombinant microorganism strategy has become necessary for further studies. Interestingly, synthetic analogs or AMP derivatives have been successfully developed based on natural peptides, generating significant improvements in their antimicrobial activity.

Most marine invertebrates are fixed to a substrate. Due to this sedentary condition, these organisms have evolved effective antimicrobials mechanisms, including AMPs (Tincu and Taylor, 2004). AMPs have been purified mainly from mollusks such as mussels, oysters, scallops and gastropods. Arenas et al. (2009) obtained and characterized an AMP native to a Chilean oyster (A. purpuratus hemocytes) via chemical synthesis. This new molecule (Ap-S) showed the presence of a polyproline type secondary structure, with a reduced number of β sheets due to the differential distribution of hydrophobic and hydrophilic residues in two well-defined zones in the N- and C-termini, compared with the native peptide (Ap). Ap-S showed no cytotoxic effect in the fish cell line CHSE-214. These findings for the newly generated Ap-S molecule make its evaluation for various biotechnological applications into a reasonable option, including its exogenous application to control important plant pathogens. In this regard, the scaled-up synthesis of this peptide becomes important for potential industrial applications.

This application describes the design and use of a chimeric gene encoding the recombinant version of Ap-S (rAps) which is expressed in E. coli strain BL21 and in Nicotiana tabacum. The results of the evaluation of rAp-S against plant pathogens, exemplified in the fungi Trichoderma harzianum, Botrytis cinerea, Fusarium oxysporum and Alternaria sp., in the Gram positive bacterium Clavibacter michiganensis and in the Gram negative bacterium Xanthomona campestris, indicated that the mid-high scale production of this peptide leads to the production of a biologically active AMP that can be successfully used with these types of phytopathogens.

The first objective of the invention is to provide a nucleotide sequence encoding recombinant antimicrobial peptides of Ap-S with antifungal and bactericidal activity.

The second objective of the invention is to provide antimicrobial peptides with a broad activity spectrum.

### BRIEF DESCRIPTION OF THE DRAWINGS

FIG. 1: Method of cloning in a suitable vector for the expression of the peptide in E. coli.

FIG. 2: Method of cloning in a suitable vector for the expression of the peptide in plants or plant cells transformed using A. tumefaciens as an agent.

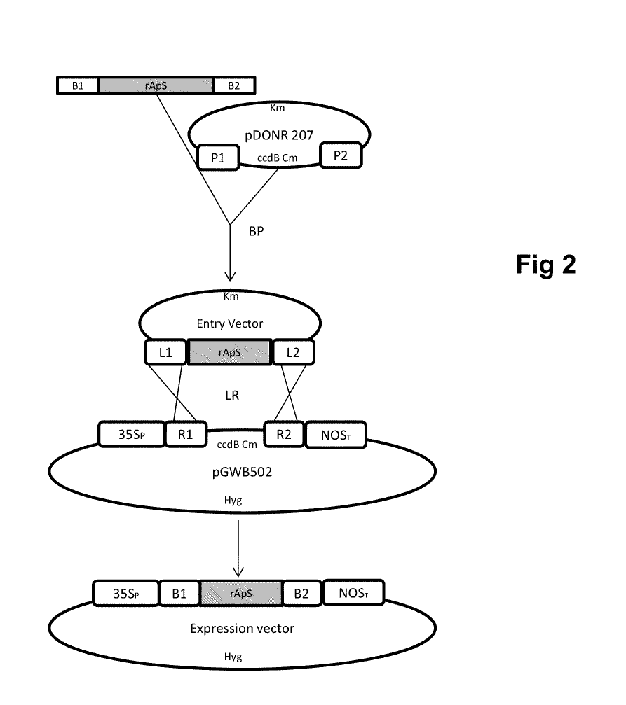

FIG. 3: Method of cloning in a suitable vector for the expression of the peptide in a plant with A. tumefaciens as a vector.

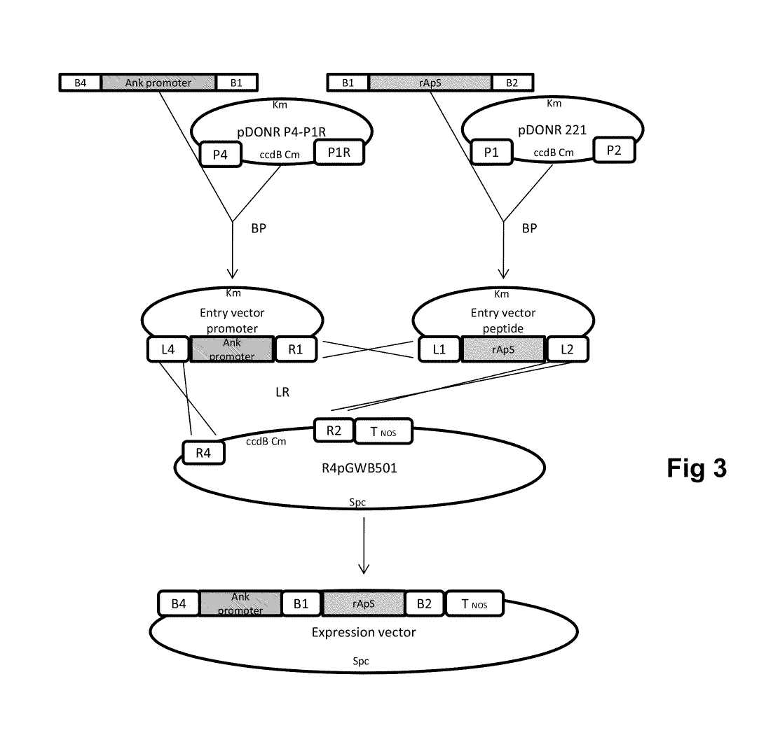

FIG. 4: Growth inhibition assays of Xanthomona campestris (G−) at different peptide concentrations. The maximum inhibition was reached between 2,000 and 4,000 μg/L.

FIG. 5: Growth inhibition assays of Clavibacter michiganensis (G+) at various peptide concentrations. The maximum inhibition was reached between 12,500 and 15,500 μg/L.

FIG. 6: Assay of the total protein extract from four lines of transgenic Nicotiana tabacum. The plates show growth inhibition halos on a lawn of T. harzianum.

### DETAILED DESCRIPTION OF THE INVENTION

The present invention relates to the generation of a nucleotide sequence encoding antimicrobial peptides.

The technical basis of the present invention is that despite knowledge of the existence of peptides with antimicrobial activity for eukaryotic organisms such as shellfish, only a small amount of peptide can be extracted from the animal tissue. This limitation has forced us to look for alternative techniques of production, such as chemical synthesis, which despite allowing the generation of greater amounts of peptide, is limited by its high cost. An alternative is the use of genetic engineering and heterologous expression systems to produce appropriate amounts in recombinant organisms. The advantage of this alternative technique is that it can produce greater amounts of peptide that will allow a more complete characterization and the actual evaluation of their biotechnological potential. However, this developmental pathway has some difficulties due to the presence of too many possible combinations of nucleotides derived from codon use to correctly define a final active peptide.

The present invention provides a synthetic gene for the AMP, Ap-S, which enables the production of large amounts of the peptide and raises its projection for use in various industrial applications, such as agriculture.

To obtain adequate amounts of the rAp-S peptide, we proceeded to design the synthetic gene and establish a cloning strategy for a synthetic sequence in an expression vector to produce synthetic peptides that are suitable for controlling fungi and bacteria and to produce synthetic peptides in quantities sufficient for use as antipathogenic agents for activities such as agriculture, veterinary practice and/or medicine.

### EXAMPLES

### Example 1

Design of the Synthetic Gene: The Ap-S cDNA was designed using the reverse-translation of the sequence of 31 amino acids (MPVGIVIAPKKSPFTAKKPGPVLSGVKAGPG) (SEQ ID N° 9) previously described based on Argopecten purpuratus hemocytes. Using GCUA software (Fuhrmann y cols., 2004) and codons from E. coli, a synthetic gene for rApS (SEQ ID N° 1) was obtained. A recognition site for the TEV-protease in the 5 ′end was added. The full oligonucleotide sequence (TEV/rApS) was synthesized at Integrated DNA Technologies, Inc. (Iowa, U.S.A.) and cloned into a pSMART vector (Lucigen, Middletown, Wis.), generating the vector pSMARTTEVrApS.

### Example 2

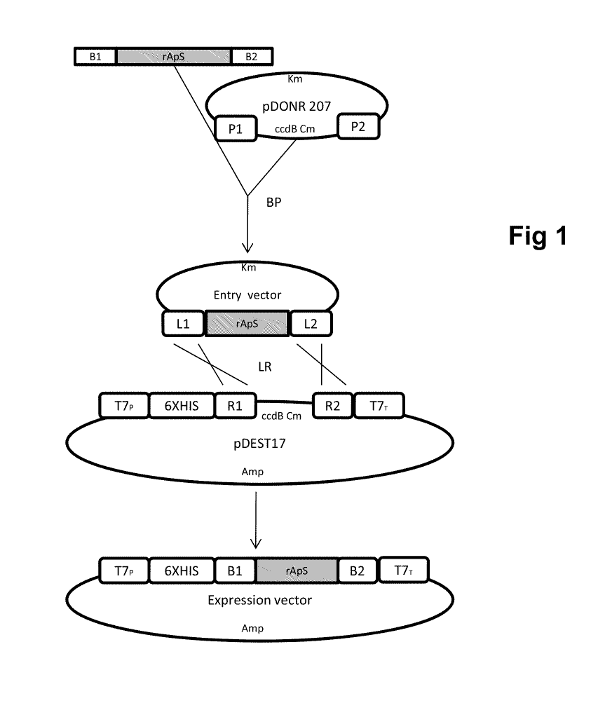

Generation of the Donor-gene Vector for Expression in E. coli: Using pSMART-TEV/rApS as a template, additional attB recombination sequences were flanked at the TEV/rApS ends using a double PCR amplification strategy. In the first step (pre-amplification), adapter-primers adpF (SEQ ID N° 2) and adpR (SEQ ID N° 3) were used to amplify a fragment of 147 bp. The PCR reaction was performed in a total volume of 50 μL containing 5 μL of Accuprime Pfx reaction mix 10× (Invitrogen, Carlsbad, Calif.), 1 μL of each primer (10 mM), 0.5 μL of AccuPrime Pfx DNA polymerase (2.5 U/μL; Invitrogen), 0.5 μL of pSMART-TEV/rApS (1:100 diluted) and 42 μL of H₂O. The thermal profile was a starting denaturation step at 95° C. for 2 min, followed by 10 cycles of 94° C. for 15 s, 55° C. for 30 s and 68° C. for 135 s. A second round of PCR was performed on 10 μL of the pre-amplified product using the primers attB1 (SEQ ID N° 4) and attB2 (SEQ ID N° 5), resulting in the synthesis of a 181 by fragment (SEQ ID N° 6) (FIG. 1a). The PCR reaction was performed in a total volume of 50 μL containing 5 μL of Accuprime Pfx reaction mix 10× (Invitrogen), 4 μL of each primer (10 μM), 0.5 μL of AccuPrime Pfx DNA polymerase (2.5 U/L; Invitrogen) and 26.5 μL of H₂O. The applied thermal profile had a starting denaturation at 95° C. for 1 min, followed by 5 cycles of 94° C. for 15 s, 45° C. for 30 s and 68° C. for 125 s, and a final round of amplification consisting of 20 cycles of 94° C. for 15 s, 55° C. for 30 s and 68° C. for 135 s. The amplification products were electrophoretically resolved in a 1% agarose gel and stained with ethidium bromide. The amplified 181 by product was excised and purified using the Qiaexll Gel Extraction Kit (Qiagen, Germany) following the protocol described by the manufacturer. The obtained DNA was used for a “BP recombination” reaction with the entry vector pDONR207 (Invitrogen, U.S.A.) (FIGS. 1b and 1c). The BP Clonase Enzyme Mix (Invitrogen) was used for the reaction according to the manufacturer's protocol. The resulting entry vector (pDNOR207-TEV/rApS) (FIG. 1c) was used for the transformation of chemically competent E. coli DH5α using standard procedures, and the recombinant clones were grown overnight in LB-agar plates supplemented with 25 mg/L gentamicin. Positive clones were confirmed by sequencing, by BsrGI enzyme restriction analysis resolved in an agarose gel, and with specific PCR reactions.

A Gateway™ LR recombination reaction was performed between pDNOR207-TEV/rApS and pDEST17 (Invitrogen, USA) (FIGS. 1d and 1e). For the reaction, LR Clonase Enzyme Mix (Invitrogen), Pstl-linearized pDNOR207-TEVrApS and pDEST17 vector were incubated according to the manufacturer's procedures. Then, 5 μL of the recombinant mixture was used for direct transformation of chemically competent E. coli BL21 (DE3) following standard procedures (Sambrook et al., 1989), and the transformed E. coli were grown overnight in LB-agar plates supplemented with carbenicillin, 100 mg/L. Primary positive clones were screened by PCR, and the final selected clone containing the pDEST17-pre-TEVrApS expression vector was confirmed by sequencing.

### Example 3

Expression and Purification of AMP in E. coli BL21 (DE3): A clone of E. coli positive for expression vector pDEST17-pre-TEV/rApS, as verified by sequencing (Macrogen Inc., Korea), was used in induction experiments of the designed recombinant peptide. An aliquot of 5 mL of the selected clone of E. coli BL21 (DE3) was prepared by incubation in LB medium supplemented with carbenicillin, 50 mg/L. The clone was cultivated overnight at 26° C. under agitation at 180 rpm. Then, four aliquots of 1 mL each were incubated in 250 mL flasks containing the same culture medium and grown for an additional 9 h. Once the cultures reached OD₆₀₀=1.8, isopropyl-b-d-thio-galactopyranoside (IPTG) was added to a concentration of 1 mM for peptide induction during an additional 3 h at four different temperatures (25° C., 26° C., 28° C., and 37° C.) and 180 rpm. The bacterial cultures were centrifuged at 12,000×g, and the supernatants were removed. The pellets were processed for peptide purification through Ni2+ charged columns using QIAexpress®Ni-NTA Fast Start (Qiagen) under denaturing conditions following the manufacturer's instructions. Four eluted fractions per culture were collected and quantified for protein content according to Bradford et al. (1976) using the Coomassie Plus (Bradford) Assay Kit (Pierce, Rockford, Ill., USA).

### Example 4

Pre-TEV/rApS Digestion and rApS Purification: The protein-enriched fraction purified from the Ni2+ affinity column was subjected to TEV peptidase processing by incubation in 12.5 μL of 20× rTEV buffer, 2.5 μL of 0.1 M DTT, 5 μL of TEV protease, and 230 μL of water. The digestion was conducted by incubation overnight at 4° C. To obtain the rApS fraction from the digestion reaction, the mixture was re-purified using the Ni2+ column, with the first eluted fraction from the column containing the peptide. The protein was quantitatively determined as previously indicated.

### Example 5

Mass Spectrometry Analysis of Purified pre-TEV/rApS and rApS: Re-purified samples of the peptides were automatically analyzed in an ABi4800 MALDI TOF/TOF mass spectrometer (Applied Biosystems, Framingham, Mass., USA) under positive ion reflector mode (ion acceleration voltage of 25 kV for MS acquisition and 1 kV for MSMS) or linear mode for peptides ≧4 kDa. The resulting spectra were stored in the ABi4000 Series Explorer Spot Set Manager. The MS and MSMS fragment ion spectra were smoothed and corrected to zero-baseline using routines embedded in the ABi4000 Series Explorer Software v3.6. External calibration was used to reach a typical mass measurement accuracy of <25 ppm.

### Example 6

Recombinant Peptide Expression in Nicotiana tabacum:

a) Construction of input vector with pDONRP4-P1 R and pDONR221. The ankyrin promoter sequence was amplified with the primers attB4 (5′-GGGGACAACTTTGTATAGAAAAGTTGTC-NNN-3′) (SEQ ID N° 7) and attB1r (5′-GGGGACTGCTTTTTTGTACAAACTTGC-NNN-3′) (SEQ ID N° 8), and the rApS sequence was amplified with attB1 (5′-NNN3 GGGGACAAGTTTGTACAAAAAAGCAGGCTTC-3′) and attB2 (5′-GGGGACCACTTTGTACAAGAAAGCTGGGTC-NNN-3′), where NNN represents the specific nucleotide sequence. The following PCR conditions were used: 5 μL of Reaction Mix 10× AccuPrime Pfx (Invitrogen), 4 μL of each primer (10 mM each), 0.5 μL of AccuPrime Pfx DNA Polymerase (2.5 U/μL; Invitrogen), 0.5 μL of vine genomic DNA and 36 μL of ddH₂O. The thermal profile was initial denaturation at 95° C. for 2 min, followed by 29 cycles with denaturation at 94° C. for 15 sec, annealing at 55° C. for 30 sec and extension at 68° C. for 2 min 15 sec. The results of the amplifications were visualized by electrophoresis on a 1% agarose gel with ethidium bromide staining (Sambrook et al., 1989). The bands corresponding to the expected size were cut and purified with Qiaexll Gel Extraction Kit (Qiagen, Germany) according to the manufacturer's instructions. The purified fragments of this reaction were used for recombination with the pDONRP4-P1 R and pDONR221 vectors (Invitrogen) with “BP Clonase” (Invitrogen) following the manufacturer's instructions. The vectors were cloned in E. coli DH5a grown in LB medium supplemented with 50 mg/L kanamycin.

b) Construction of expression vector with R4pGWB401. For the construction of the plant expression vector, the vectors pDONRP4-P1R-Ankyrin and pDONR221-rApS containing the regulatory and coding sequences, respectively, were recombined with vector R4pGWB401 (Nakagawa et al., 2009) using “LR Clonase” (Invitrogen). The resulting vector was cloned in E. coli DH5α grown in LB medium supplemented with 50 mg/L spectinomycin.

c) Construction of expression vector with pGWB502. For the construction of the plant expression vector, the pDONR207-TEV/rApS vector containing the chimeric gene and the target vector pGWB502 (Nakagawa et al., 2007) were recombined using “LR Clonase” (Invitrogen). The resulting vector was cloned in E. coli DH5α and grown in LB medium supplemented with 50 mg/L spectinomycin.

d) Transformation of tobacco: For obtaining genetically modified tobacco plants with the expression vector, the protocol of Sparkes et al. (2006) was used. Young leaves of approximately 4-6 weeks were infiltrated with a solution containing acetosyringone 1 M, sodium orthophosphate dodeca-hydrate (Na₃PO₄.12H₂O) 2 mM, MES (2-[N-morpholin]-ethanesulfonic acid; Phytotechnology Labs) 50 mM, D-glucose 25 mM and A. tumefaciens 3101 (OD₆₀₀=0.3) harboring the vector to evaluate the plant material. After 72 hours of infiltration, the leaves of the plant were removed, sterilized with a 10% solution of sodium hypochlorite, washed with sterile ddH₂O and cut into square pieces of approximately 1 cm². The fragments were cultured in Petri dishes containing 4.43 g/L germination medium (MS-S) of Murashige and Skoog (1962), supplemented with vitamins (Phytotechnology Labs, USA), 0.8% (w/v) agar TC (Phytotechnology Labs), 3.0% (w/v) sucrose (Phytotechnology Labs), 0.1 mg/L indole butyric acid (IBA, Sigma Aldrich, USA), 0.8 mg/L 6-benzylaminopurine (BAP; Sigma Aldrich), 200 mg/L carbenicillin (Sigma Aldrich), 200 mg/L timentin (Phytotechnology Labs) and 300 mg/L kanamycin (Phytotechnology Labs). The explants were maintained in MS-S to sprout, which occurred in approximately 3 weeks. Subsequently, the shoots were excised and cultured for two weeks in Petri dishes with 4.43 g/L rooting medium (MS-R) of Murashige and Skoog supplemented with vitamins, 0.8% (w/v) TC agar, 3.0% (w/v) sucrose, 0.5 mg/L indole butyric acid (IBA), 200 mg/L carbenicillin, 200 mg/L timentin and 300 mg/L kanamycin. Finally, regenerated plantlets were planted separately in flasks (150 mL) containing base MS medium (4.43 g/L of Murashige and Skoog supplemented with vitamins, 0.8% [w/v] national agar (Veronica Sepulveda, Chile) and 3.0% [w/v] sucrose). The plants were maintained in this medium until evaluation.

### Example 7

Functionality Tests of Peptides Produced in Bacteria and Plants:

a) Evaluation of Peptides obtained in E coli: Isolates from Botrytis cinerea, Fusarium oxysporum, Trichoderma harzianum and Alternaria spp. were treated with the purified fractions of the digested and non-digested peptides (pre-TEV/rApS and rApS) derived from the selected recombinant clone of E. coli (Example 3).

The potential antifungal abilities of both peptides were evaluated by analysis of the growth patterns of these fungi on agar PDA (potato-dextrose) in Petri dishes supplemented with protein extracts at 500 μg/L (162 nM) and 250 μg/L (81 nM), respectively.

The control growth assays were performed on PDA dishes without peptides. The growth patterns were evaluated according the amount of time required for the fungi to reach the dish walls in the control plates (10 days after inoculation in darkness at room temperature). Structural images of the fungi hyphae patterns were obtained using a Olympus® microscope (Center Valley, Pa., USA), and spores were counted using a Neubauer camcorder (Brand®, Wertheim, Germany) at 40×. The data were subjected to analyses of variance, and averages were separated by the least significant difference test (LSD) at the 5% level of significance using Statgraphics Centurion XV (Manugistics Inc., Rockville, Md., USA). Petri dishes containing grown fungi were scanned and digitalized for statistical processing. The area of the mycelia was quantified using Gel-Pro Analyzer 4.0 (Media Cybernetics, Minneapolis, Minn., USA), and surface values in pixels were transferred to Excel (Microsoft Corporation, USA) for analysis. The average value of the colored haloes was calculated using scanned images of five repetitions on the tenth day after inoculation. The collected data were subjected to variance analysis, and the averages separated by LSD at the 5% level of significance. The confrontation results of the peptides are shown in tables 1 and 2. Table 1 shows the spore count. Table 2 shows the quantification of the area of the mycelium in pixels.

TABLE 1

| | Spore count | | | | | | | | | | | |
|---|---|---|---|---|---|---|---|---|---|---|---|---|
| Purified | Trichoderma | | | | | | | | | Fusarium | | |
| peptide | harzianum | | | Botrytis cinerea | | | Alternaria sp | | | oxysporum | | |
| fraction | average | DE | LSD | average | DE | LSD | average | DE | LSD | average | DE | LSD |
| rApS | 5.0 × 10⁶ | 1.0 × 10⁵ | a | 1.02 × 10⁶ | 2.31 × 10⁵ | A | 0 | 0 | a | 7.6 × 10⁵ | 8.98 × 10⁴ | a |
| Pre- | 1.40 × 10⁷ | 1.55 × 10⁶ | b | 5.98 × 10⁵ | 8.92 × 10⁴ | A | 1.82 × 10⁶ | 2.33 × 10⁵ | b | 1.17 × 10⁶ | 7.27 × 10⁴ | b |
| TEV/rApS | | | | | | | | | | | | |
| Control | 2.14 × 10⁷ | 1.57 × 10⁶ | c | 9.17 × 10⁶ | 4.93 × 10⁵ | B | 3.15 × 10⁶ | 9.29 × 10⁴ | c | 1.76 × 10⁶ | 1.31 × 10⁵′ | c |

TABLE 2

| Petri | Central Area (pixels) | | | | | | | | | | | |
|---|---|---|---|---|---|---|---|---|---|---|---|---|
| dish/agar | Trichoderma | | | | | | | | | Fusarium | | |
| PDA | harzianum | | | Botrytis cinerea | | | Alternaria sp | | | oxysporum | | |
| plus: | average | DE | LSD | average | DE | LSD | average | DE | LSD | average | DE | LSD |
| rApS | 85718 | 12155 | a | 170781 | 69412 | A | 412403 | 14385 | a | 511699 | 55046 | a |
| Pre- | 623533 | 95785 | b | 235089 | 24328 | A | 430672 | 7665 | a | 498691 | 78927 | a |
| TEV/rApS | | | | | | | | | | | | |
| Control | 820412 | 42611 | c | 824804 | 53429 | B | 460848 | 18115 | b | 670810 | 98093 | b |

### Example 8

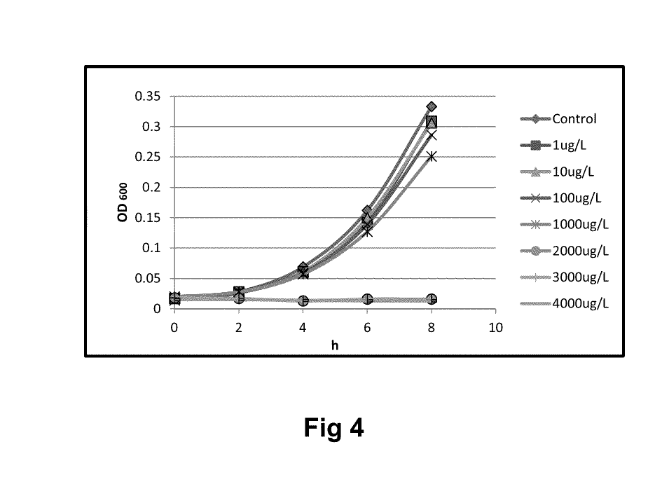

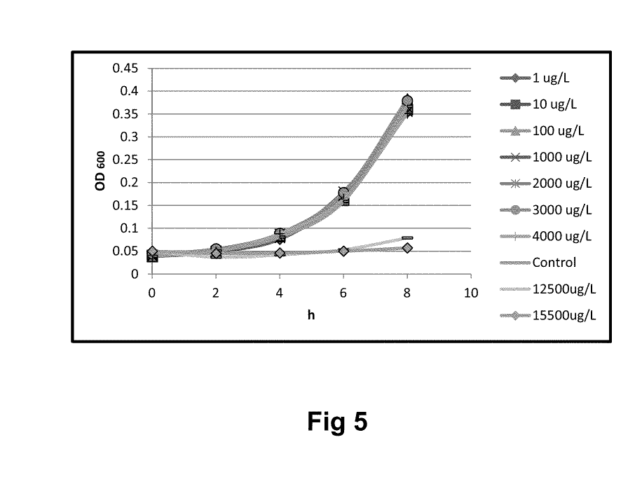

Growth Inhibition Assays at Different Concentrations of Peptide rApS: The inoculation of 5 mL of LB was performed with 100 μL of bacteria (Xanthomona campestris and Clavibacter michiganensis), followed by overnight culturing. The absorbance was quantified at OD₆₀₀. Once the Abs was quantified, dilutions were made to obtain an Abs to inoculate each tube with the same concentration of bacteria with an initial OD₆₀₀ of 0.04. Different peptide concentrations were used. Each tube was inoculated with an rApS peptide concentration, as shown in Tables 3 and 4. A final volume of 10 mL in a 50 mL tube was used. In addition, a control without peptide was used for each bacterium. The kinetics of the process was followed for 8 h from time zero, determining the Abs every 2 h. The sample was 1 mL, as shown in FIGS. 4 and 5. The data were logarithmically transformed for the exponential phase of kinetics, and a linear regression was performed on the transformed data. The slope of the regression is the specific growth rate of the bacteria: p (h-1). The p (specific growth rate) was compared by LSD, and the data were interpreted, as shown in Tables 3 and 4.

TABLE 3
Xanthomona campestris (G−)

| | Xanthomona campestris | | |
|---|---|---|---|
| Tube | μ (h⁻¹) | SD | LSD |
| Control | 0.385 | 0.011 | a |
| 1 μg/L | 0.371 | 0.011 | a |
| 10 μg/L | 0.375 | 0.012 | a |
| 100 μg/L | 0.386 | 0.007 | a |
| 1 mg/L | 0.329 | 0.014 | b |
| 2 mg/L | −0.015 | 0.002 | c |
| 3 mg/L | −0.012 | 0.002 | c |
| 4 mg/L | −0.019 | 0.002 | c |

TABLE 4
Clavibacter michiganensis (G+)

| | Clavibacter michiganensis | | |
|---|---|---|---|
| Tube | μ (h⁻¹) | SD | LSD |
| Control | 0.072 | 0.011 | a |
| 1 μg/L | 0.070 | 0.008 | a |
| 10 μg/L | 0.069 | 0.010 | a |
| 100 μg/L | 0.075 | 0.010 | a |
| 1 mg/L | 0.069 | 0.006 | a |
| 2 mg/L | 0.069 | 0.009 | a |
| 3 mg/L | 0.073 | 0.009 | a |
| 4 mg/L | 0.066 | 0.009 | a |
| 12.5 mg/L | 0.009 | 0.001 | b |
| 15.5 mg/L | 0.003 | 0.0003 | b |

b) Evaluation of the rAp-S produced in Nicotiana tabacum

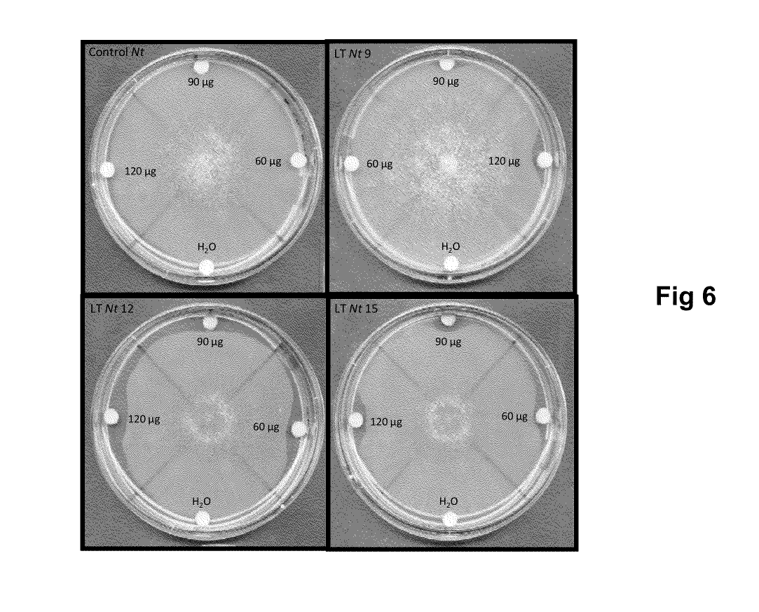

For the evaluation of the rAp-S produced in Nicotiana tabacum, total protein was extracted from 14 transgenic lines, and 30 μg of total protein from each plant was spread on a Petri plate containing PDA. Each plate was inoculated with a suspension of 2.0×10⁹ spores of T. harzianum as the growth inhibition indicator. After 7 d, the growth inhibition was determined visually, and no biological or statistical analysis was performed if there was any plaque inhibition. The three top lines that generated some degree of growth inhibition were selected, and a further test was performed with 60, 90 and 120 μg of total protein in the same plate. The plate was divided into four equal areas, and in each area, a Whatman paper disk was placed on the edge with the required peptide amount or a water control. All peptide amounts (60, 90 and 120 μg) were added to a volume of 100 μL to ensure that the experiment was more homogeneous. Each plate was inoculated with a spore suspension of 2.0×10⁹ of T. harzianum in the center. After 3 days, the inhibition area was determined with the program Pro Analyzer Gel using the disk peptide areas where the fungus did not grow (FIG. 6). The areas of inhibition, in pixels, of the transgenic lines (TL) of Nicotiana tabacum (Nt) were determined: TL Nt 9, TL Nt 12, TL Nt 15 and Control Nt (untransformed). Each line, including the Control Nt, was analyzed separately. In the LSD analysis, the different amounts of total protein (60, 90, and 120 μg and water) were compared. The Control Nt and water controls for each TL Nt showed no inhibition of T. harzianum (Table 5).

TABLE 5
Total protein extracts from tobacco.

| | Inhibition Area of Trichoderma harzianum (pixels) | | | | | | | | | | | |
|---|---|---|---|---|---|---|---|---|---|---|---|---|
| Petri | Control Nt | | | LT Nt 9 | | | LT Nt 12 | | | LT Nt 15 | | |
| Dish | Average. | SD | LSD | Average | SD | LSD | Average | SD | LSD | Average | SD | LSD |
| water | * | * | ** | * | * | ** | * | * | ** | * | * | ** |
| 60 μg | * | * | ** | 221.67 | 0.47 | a | 3416.5 | 94.5 | a | 1048.67 | 26.4 | a |
| 90 μg | * | * | ** | 516 | 108 | b | 3340 | 37 | a | 1930.5 | 36.5 | b |
| 120 μg | * | * | ** | 605 | 169.41 | b | 4185.5 | 271.5 | b | 2741 | 20 | c |

Inhibition of T. harzianum with total protein extracts from transgenic lines (LT) of Nicotiana tabacum (Nt) at increasing amounts (60, 90 and 120 μg) and a water control. The Control Nt was a non-transgenic plant.
*No inhibition;
**No statistical analysis.

These experimental examples were exemplifying and not limiting. Although the yields obtained and the activity of the recombinant peptide were successful in their objective of producing rAp-S active against economically relevant pathogens using bacteria and plants as sources of expression, the present invention does not exclude the potential use of this chimeric gene to express the said AMP in additional expression systems, such as yeast. The use of a medium/large scale of such peptides is desirable for the control of plant pests.

Optionally and as a non-limiting method, the methodology to be used for the implementation of the total protein extract of N. tabacum can be performed together with a suitable carrier.

Protein extraction was performed with a basic extraction buffer consisting of 1 M sodium chloride, 0.1 M sodium acetate, 1% PVP, 10 mM β-mercaptoethanol, 0.25% v/v Triton X-100, and 20% glycerol. This total extract can be diluted to the required concentration in ddH₂O (pH between 4 and 5) and sprayed onto the plants. The plants can also be treated using alternative means.

The extracts of total protein from E. coli were prepared from the method described in Example 3 and then diluted in ddH₂O (pH between 4 and 5) to the concentration required for spraying.

These preparations are suitable for the direct application to crops, plants, plant parts, roots and/or soil that is healthy or infested with fungi and/or phytopathogenic bacteria.

After obtaining the protein extracts from both E. coli and N. tabacum, a lyophilization step can be incorporated, which gives stability to the extract and allows storage at room temperature. Additionally, this step will facilitate the commercialization of the product.

In short, if the extract is obtained by synthesis in E. coli, the process is scaled to high volume bioreactors, ball or disc grinder, continuous or cake centrifuge, extract stripper column and a final lyophilizer to process this volume.

In the case that the process selected for obtaining the extract uses N. tabacum, an extension of cropland that allows the required demand to be met, ball grinder, continuous or cake centrifuge and a lyophilizer able to process the final high volume should be considered.

1. A chimeric nucleotide sequence encoding chimeric peptides with antimicrobial activity, wherein said sequence is a synthetic DNA sequence obtained from a back-translation of the amino acid sequence of peptide Ap-S of the Argopecten purpuratus scallop.

2. A chimeric nucleotide sequence encoding peptides with antimicrobial activity according to claim 1, wherein the DNA sequence of the peptide comprises the DNA sequence of SEQ ID No. 1

3. A chimeric nucleotide sequence encoding peptides with antimicrobial activity according to claim 2, wherein the sequence of SEQ ID No. 1 is amplified by the primers of sequence SEQ ID No. 4 and SEQ ID No. 5 and said nucleotide sequence adds the attb recombination sites, which enable the insertion of the amplification product of SEQ ID No. 6 in a donor plasmid containing the attp recombination sites, as shown in FIG. 1.

4. A nucleotide sequence according to claim 3, wherein said sequence has been designed for expression in bacteria, plants or plant cells and encodes a peptide having antimicrobial characteristics.

5. Use of the nucleotide sequences SEQ ID No. 1 and SEQ ID No. 6 to confer resistance or tolerance to plant pathogenic fungi or bacteria on a plant, comprising the introduction of said sequences into plant materials.

6. The nucleotide sequences of claim 5, wherein said nucleotide sequences are designed through codon usage to obtain the same peptide in E. coli and N. tabacum.

7. Recombinant peptides derived from the sequences SEQ ID No. 1 and SEQ ID No. 6, wherein they are obtained from the translation of SEQ ID No. 1 and SEQ ID No. 6 in E. coli and N. tabacum.

8. Recombinant peptides according to claim 7, wherein one end contains the histidine peptide residues necessary for the expression and/or purification of said recombinant peptides.

9. A plant, plant cell, seed or transformed plant material that produces the peptide sequence derived from SEQ ID No. 1 and SEQ ID No. 6.
